(19) United States
(12) Patent Application Publication
(10) Pub. No.: US 2013/0310623 A1
(43) Pub. Date: Nov. 21, 2013

(54) AMINE GAS TREATMENT SOLUTIONS

(71) Applicants: Michael SISKIN, Westfield, NJ (US); Robert B. FEDICH, Long Valley, NJ (US); Michel A. DAAGE, Hellertown, PA (US)

(72) Inventors: Michael SISKIN, Westfield, NJ (US); Robert B. FEDICH, Long Valley, NJ (US); Michel A. DAAGE, Hellertown, PA (US)

(73) Assignee: ExxonMobil Research and Engineering Company, Annandale, NJ (US)

(21) Appl. No.: 13/471,758

(22) Filed: May 15, 2012

(51) Int. Cl. C07C 7/11; C09K 3/00

(52) U.S. Cl. CPC C07C 7/11; C09K 3/00
USPC 585/863; 252/184; 585/860

(57) ABSTRACT

A process for the selective absorption of acidic components from normally gaseous hydrocarbon mixtures using an aqueous amine absorbent solution comprising an antioxidant and a non-detergent co-solvent for the amine and the antioxidant.

### FIELD OF THE INVENTION

The present invention relates to the absorption of acidic gases from a mixed gas streams containing acidic and non-acidic components.

### BACKGROUND OF THE INVENTION

The treatment of gases and liquids containing acidic gases such as CO₂, H₂S, CS₂, HCN, COS and sulfur derivatives of C₁ to C₄ hydrocarbons with amine solutions to remove these acidic gases is well established. The amine usually contacts the acidic gases and the liquids as an aqueous solution containing the amine in an absorber tower with the aqueous amine solution passing in countercurrent to the acidic fluid. Typical processing operations use common amine sorbents such as: monoethanolamine (MEA), diethanolamine (DEA), methyldiethanolamine (MDEA), diisopropylamine (DIPA), or hydroxyethoxyethylamine (DGA). The liquid amine stream containing the sorbed acid gas is typically regenerated by desorption of the sorbed gases in a separate tower with the regenerated amine and the desorbed gases leaving the tower as separate streams. The various gas purification processes which are available are described, for example, in Gas Purification, Fifth Ed., Kohl and Neilsen, Gulf Publishing Company, 1997, ISBN-13: 978-0-88415-220-0.

A number of practical problems arise during the operation of these amine units. For example, the gases to be treated may be at relatively high temperatures and contain molecular oxygen or other potential oxidants which may react with the amine treating agent(s) to degrade it and so remove it from the available inventory, reducing the effective rating of the unit. Another problem is foam generation, especially in the aqueous amine solutions which are the most typical in most units: the amines themselves are basic and tend to lower the surface tension of the solution and facilitate the generation of foams as the solution is agitated by the pump-induced circulation in the unit and the counterflow of the incoming gas. Yet another problem is corrosion, not from the amines since all common amines and ethanolamines such as MEA, DEA, MDEA and DGA are essentially non-corrosive to mild steels, but, rather from the acidic gas streams, e.g., carbon dioxide or hydrogen sulfide, and/or from the reaction products with the amines and amine degradation products.

Amines react with carbon dioxide to yield compounds that can increase corrosion activity by a factor of 10 to 100 over the same solution without these reaction products and these compounds may, in processing high concentrations of carbon dioxide, be the primary cause of corrosion. Corrosive degradation products are formed in most amine solutions used for carbon dioxide removal including MEA, DEA, MDEA, blended solvents of DEA and MDEA and formulated solvents based on MDEA that use an ethanolamine as a promoter to improve CO₂ absorbtion. Ethylenediamine oxidative degradation products accumulate in the amine solution resulting in higher corrosion rates as the amine solution ages. When the ethanolamines react with CO₂, the main non-aqueous reaction product is an ethanolamine carbamic acid; the carbamic acid of the primary and secondary ethanolamines (MEA, DEA and MDEA), undergo further reactions which over time and in the presence of oxygen and/or heat, lead to irreversible degradation of the ethanolamines. The semi-reversible reaction of ethanolamine carbamic acid with CO₂ forms 5-member ring compounds (oxazolidones) which, with sufficient heat and stripping efficiency, partially revert to the parent ethanolamine but, unfortunately, also cause the oxazolidones to react irreversibly to form substituted ethylenediamine compounds that are powerful metal chelants that remove iron, nickel and chromium from alloys and protective scales. The chelation of metal ions by these compounds which occurs particularly when the ratio of CO₂ to H₂S is greater than 10, destroys protective iron oxides and sulfides scales to form water soluble chelated metal species. To counter these problems, various additives including antioxidants, defoamants, corrosion inhibitors are conventionally added to the units. A number of additives are commercially available to counter corrosion by these species. One example of a corrosion inhibitor is Max-Amine GT 741-C™ (trademark of General Electric Company) which is stated to be useful for reducing the corrosion caused by carbon dioxide and organic acids in various amine/ethanolamine treatment systems.

Oxidation of the amine treating agents by the incoming gas is not a major problem in the processing of natural gas streams since their composition does not include oxidants but other gas streams, e.g., flue gas, may contain oxygen or other potential oxidants that will degrade the amines and alkanolamines and produce reaction products which themselves may be corrosive and which, in any event reduce the amount of treating agent available for processing the gas. The addition of anti-oxidant agents to the treatment solution may be effective to inhibit oxidative degradation of the active agent. While certain antioxidants are water-soluble to varying extents and can be dissolved in effective amounts (effective to inhibit oxidation) in the treatment solution, others may not be. Phenolic antioxidants as well as certain diarylamine antioxidants typically used in quantities of 1-500 wppm, may be sufficiently soluble in water to act as effective antioxidants in aqueous amino treatment solutions but other potentially useful antioxidants are likely to be insoluble or not soluble to a sufficient degree. These materials are likely to be less effective in inhibiting oxidation of the amino treating agent since they will be suspended as discrete liquid particles in the circulating solution and therefore have to cross the phase barrier into the aqueous phase in order to become fully effective.

While the addition of surface active detergents to the solution would improve the effectiveness of the less-soluble or insoluble antioxidants, it is unlikely to resolve the problem altogether and may also create new difficulties, especially of foaming. As noted above, foam formation is an ever present operational problem and usually defoamants such as GE-Toshiba SAG-7133 are present in the solution to reduce its onset. While it is possible to use a defoamant as well as a surface active detergent so as to have one offsetting the effect of the other, it would be preferable to maintain antioxidants and other less-soluble additives in solution with the amine without the necessity of resorting to the use of surface active agents to maintain the solubility of the additive.

### SUMMARY OF THE INVENTION

According to the present invention we envisage that non-detergent materials will be used to bring the antioxidant into solution in the water with the amine by acting as a co-solvent for the antioxidant and the amine. In this way, the antioxidant will be readily available to exert its effect on the amine. The co-solvent may be formulated as a pre-mixed package with the amine absorbent and the antioxidant or, alternatively, mixed with the amine at the site of use.

The present invention therefore provides, in one of its various aspects a process for the selective absorption of normally gaseous acidic components from hydrocarbon gas mixtures containing both the acidic component and gaseous non-acidic components in which an aqueous amine absorbent solution is circulated in a cyclic amine absorption gas purification unit to absorb acidic gases from the hydrocarbon gas in an absorption tower and to desorb acidic gases in a regeneration tower to produce a stream of purified hydrocarbon gas and at least one stream of acidic gas removed from the hydrocarbon gas, the aqueous amine absorbent solution also comprising an antioxidant and a co-solvent for the amine and the antioxidant.

In another aspect, the invention provides a composition of matter comprising (i) an amine absorbent for the selective absorption of normally gaseous acidic components from hydrocarbon gas mixtures containing the acidic component and gaseous non-acidic components, (ii) an antioxidant and (iii) a co-solvent for the amine and the antioxidant.

### DETAILED DESCRIPTION

### Selective Absorption Process

The selective absorption of the acidic gases from the gas mixture or stream is typically carried out by contacting the gaseous stream with the absorbent solution in any suitable contacting vessel. In such processes, the normally gaseous mixture from which the acid gases are to be selectively removed may be brought into intimate contact with the absorbent solution using conventional equipment such as a tower or vessel packed with, for example, rings or with sieve plates, or a bubble reactor.

Typically, the absorption is conducted by feeding the normally gaseous mixture at the lower end of the absorption tower while fresh absorbent solution is fed into the upper region of the tower. The gaseous mixture, freed largely from the acidic components, emerges from the upper portion of the tower, and the loaded absorbent solution, containing the absorbed gases, leaves the tower near or at its bottom. The inlet temperature of the absorbent solution during the absorption step is preferably in the range of from about 20° to about 100° C., and more preferably from 40° to about 60° C. Pressures may vary widely; typical pressures are between 25 and 14,000 kPag, preferably 100 to 10,000 kPag, and most preferably 150 to 7,000 kPag in the absorber (about 4 to 2030 psig, preferably 15 to 1450 psig, more preferably 22-10, 150 psig). The contacting takes place under conditions such that the acidic components are selectively absorbed by the amine absorbent solution. It is possible to adjust absorption conditions and apparatus to minimize the residence time of the liquid in the absorber to reduce CO₂ pickup while at the same time maintaining sufficient residence time of gas mixture with liquid to absorb a maximum amount of the H₂S gas if using an absorbent which has selectivity for H₂S in preference to CO₂, such as those described in U.S. Pat. Nos. 4,405,583; 4,405,585, 4,471,138, 4,894,178 and U.S. Patent Publication 2010/0037775, to which reference is made for a full description of these materials, their synthesis and their use in selective acidic gas separation processes.

The amount of liquid required to be circulated to obtain a given degree of acid gas separation will depend on the chemical structure and basicity of the amino absorbent and on the partial pressure of the respective acidic component in the feed gas. Gas mixtures with low partial pressures such as those encountered in thermal conversion processes will require less liquid under the same absorption conditions than gases with higher partial pressures such as shale oil retort gases.

In a typical procedure for the selective H₂S removal in the presence of CO₂, a countercurrent contact of the gaseous mixture containing H₂S and CO₂ with the aqueous solution of the amino compounds is maintained in a column containing a plurality of trays at a low temperature, e.g., below 45° C., and at a gas velocity of at least about 0.1 m/sec (based on “active” or aerated tray surface), depending on the operating pressure of the gas. A tray column having fewer than 20 contacting trays, with, e.g., 4-16 trays is typically employed.

After contacting the gas mixture, the absorbent solution becomes saturated with the separated acidic components or components and may be at least partially regenerated so that it can be recycled back to the absorber. As with absorption, the regeneration may take place with the absorbent solution in a single liquid phase. Regeneration or desorption of the acid gases from the absorbent solution may be accomplished by conventional means such as pressure reduction of the solution or increase of temperature to a point at which the absorbed gases flash off, or stripping by passing the solution into a vessel in which a stream of an inert gas such as air or nitrogen or preferably steam is passed upwards through the vessel. The temperature of the solution during the regeneration step will typically be in the range from about 50° to about 170° C., and preferably from about 80° to 120° C., and the pressure of the solution on regeneration will typically range from about 5 to 500 kPag, preferably 5 to about 250 kPag. The absorbent solution, after being cleansed of at least a portion of the dissolved component(s), may be recycled back to the absorbing vessel. Makeup absorbent may be added a needed.

Further details of the selective absorption process may be found in U.S. Pat. Nos. 4,405,583; 4,405,585; 4,471,138; 4,894,178 and U.S. Patent Publication 2010/0037775, to which reference is made for a description of such separation processes, especially processes which will achieve selective separation of H₂S from gas mixtures containing CO₂.

### Aminoether Absorbents

While the proposed transport scheme is applicable to the broad class of liquid amines which may be used for the absorption of acidic gases such as H₂S and CO₂ from gas streams such a natural gas, syngas etc, the preferred amine sorbents are those which may be used for the selective sorption of H₂S from acidic gas streams which are mixtures of H₂S with CO₂ and other acidic gases such as CS₂, HCN, COS and sulfur derivatives of C₁ to C₄ hydrocarbons. This preferred class of aminoethers is represented by the derivatives of diethylene glycol or polyethylene glycols which contain severely sterically hindered amino groups as well as by their corresponding derivatives derivatized on the alcohol group to form the corresponding ether or ester derivatives and their corresponding sulfonate and phosphonate salts. In general, the preferred severely sterically hindered aminoether derivatives will have a cumulative Es (Taft steric hindrance constant) value greater than 1.75 (see below for further explanation of this constant and its calculation).

Preferred examples of these aminoethers are disclosed in U.S. Pat. Nos. 4,405,583; 4,405,585, 4,471,138, 4,894,178 and U.S. Patent Publication 2010/0037775, to which reference is made for a full description of these materials, their synthesis and their use in selective acidic gas separation processes. Their disclosures are summarized below for convenience.

U.S. Pat. No. 4,405,583: The hindered diamino ethers disclosed in this patent are defined by the formula:

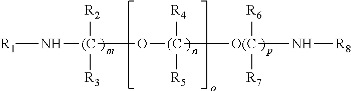

where R¹ and R⁸ are each C₁ to C₈ alkyl and C₂ to C₈ hydroxyalkyl groups, R², R³, R⁴, R⁵, R⁶, and Ware each hydrogen, C₁-C₄ alkyl and hydroxyalkyl groups, with certain provisos to define the adequately hindered molecule and m, n, and p are integers from 2 to 4 and o is zero or an integer from 1 to 10. A typical diamino ether of this type is 1,2-bis(tert-butylaminoethoxy) ethane, a diamino derivative of triethylene glycol.

U.S. Pat. No. 4,405,585: The hindered amino ether alcohols disclosed in this patent are defined by the formula:

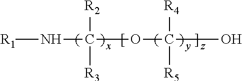

where R¹ is C₁-C₈ primary alkyl and primary C₂-C₈ hydroxyalkyl, C₃-C₈ branched chain alkyl and branched chain hydroxyalkyl and C₃-C₈ cycloalkyl and hydroxycycloalkyl, R², R³, R⁴ and R⁵ are each hydrogen, C₁-C₄ alkyl and C₁-C₄ hydroxyalkyl radicals, with the proviso that when R1 is a primary alkyl or hydroxyalkyl radical, both R² and R³ bonded to the carbon atom directly bonded to the nitrogen atom are alkyl or hydroxyalkyl radicals and that when the carbon atom of R¹ directly bonded to the nitrogen atom is secondary at least one of R² or R³ bonded to the carbon atom directly bonded to the nitrogen atom is an alkyl or hydroxyalkyl radical, x and y are each positive integers from 2 to 4 and z is an integer from 1 to 4. Exemplary compounds of this type include the amino ether alcohol tert-butylaminoethoxyethanol, a derivative of diethylene glycol.

U.S. Pat. No. 4,471,138: This patent discloses the desirability of using a combination of a diamino ether with an aminoether alcohol. The two compounds are represented by the respective formulae:

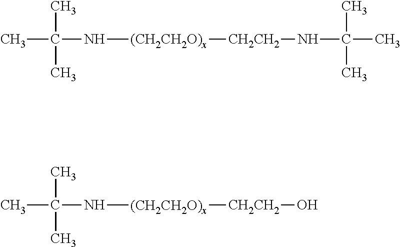

where x is an integer ranging from 2 to 6. This mixture can be prepared in the novel one-step synthesis, by the catalytic tertiary butylamination of a polyalkenyl ether glycol, HO—(CH₂CH₂O)x-CH₂CH₂—OH, or halo alkoxyalkanol. For example, a mixture of bis-(tert-butylaminoethoxy)ethane (BTEE) and ethoxyethoxyethanol-tert-butylamine (EEETB) can be obtained by the catalytic tert-butylamination of triethylene glycol. The severely hindered amine mixture, e.g., BTEE/EEETB, in aqueous solution can be used for the selective removal of H₂S in the presence of CO₂ and for the removal of H₂S from gaseous streams in which H₂S is the only acidic component, as is often the case in refineries.

U.S. Pat. No. 4,894,178: A specific combination of diamino ether and aminoalcohol represented by the respective formulae:

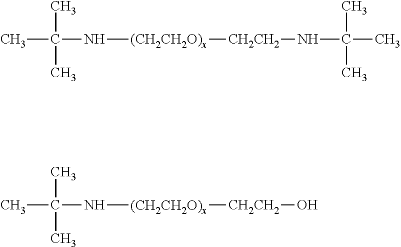

with x being an integer ranging from 2 to 6 and the weight ratio of the first amine to the second amine ranging from 0.23:1 to 2.3:1 and preferably 0.43 to 2.3:1. This mixture can be prepared in the one-step synthesis, by the catalytic tert-butylamination of the corresponding polyalkenyl ether glycol, for example, by the catalytic tert-butylamination of triethylene glycol. This mixture is one of the preferred absorbents for use in offshore gas processing.

US 2010/0037775: The reaction of a polyalkenyl ether glycol with a hindered amine such as tert-butylamine to form useful aminother absorbents is improved by the use of an alkoxy-capped glycol in order to preclude the formation of an unwanted cyclic by-product, tert-butyl morpholine (TBM). A preferred capped glycol is methoxy-triethylene glycol although the ethoxy-, propoxy- and butoxy homologs may also be used. The reaction between triethylene glycol and tert-butylamine is shown to produce a mixture of bis-(tert-butylaminoethoxy) ethane and tert-butylaminoethoxyethoxyethanol in a weight ratio of about 65-67%:33% for a total yield of about 95% of the mixture over an extended reaction time while the reaction with the alkoxy-capped glycol produces the mono-amino reaction product in comparable yield after a significantly shorter reaction time.

The aminoether compounds may be used in conjunction with other related materials such as an amine salt as described in U.S. Pat. No. 4,618,481. The severely sterically hindered amino compound can be a secondary amino ether alcohol or a disecondary amino ether. The amine salt can be the reaction product of the severely sterically hindered amino compound, a tertiary amino compound such as a tertiary alkanolamine or a triethanolamine, with a strong acid, or a thermally decomposable salt of a strong acid, i.e., ammonium salt or a component capable of forming a strong acid.

Similarly, U.S. Pat. No. 4,892,674 discloses a process for the selective removal of H₂S from gaseous streams using an absorbent composition comprising a non-hindered amine and an additive of a severely-hindered amine salt and/or a severely-hindered aminoacid. The amine salt is the reaction product of an alkaline severely hindered amino compound and a strong acid or a thermally decomposable salt of a strong acid, i.e., ammonium salt.

A preferred class of aminoethers for offshore application is defined by the formula:

R¹—NH—[CnH2n—O-]ₓ—OY

where R¹ is a secondary or tertiary alkyl group of 3 to 8 carbon atoms, preferably a tertiary group of 4 to 8 carbon atoms, Y is H or alkyl of 1 to 6 carbon atoms, n is a positive integer from 3 to 8 and x is a positive integer from 3 to 6. The preferred R¹ group is tertiary butyl and the most preferred amino ethers are those derived from triethylene glycol (n is 2, x is 3). When Y is H, the amino ether is an amino ether alcohol such as tert-butylamino ethoxyethoxyethanol, derived from triethylene glycol; when Y is alkyl, preferably methyl, the amino ether is an alkoxy amino ether, with preference for tert-butylamino methoxy-ethoxyethoxyethanol. The monoamino ethers may be used in blends with diamino ethers in which the terminal OH group of the ether alcohol or the terminal alkoxy group of the alkoxy amino ether is replaced by a further hindered amino group as expressed in the formula:

R¹—NH—[CnH₂n—O-]x—NHR²

where R¹, n and x are as defined above and R², which may the same or different to R¹, is a secondary or tertiary alkyl group of 3 to 8 carbon atoms. A preferred diamino ether of this type is bis-(t-butylamino ethoxy) ethane which may conveniently be used as a mixture with tert-butylamino methoxy-ethoxyethoxyethanol in a weight ratio of about 65-67 wt %:33-35 wt % or 33.3-35 wt %:65-66.7 wt %.

The severely sterically hindered secondary aminoether mentioned above are characterized by acyclic or cyclic moieties attached to the amino nitrogen atom(s). The term “severely sterically hindered” signifies that the nitrogen atom of the amino moiety is attached to one or more bulky carbon groupings. Typically, the severely sterically hindered aminoether alcohols have a degree of steric hindrance such that the cumulative Eₛ value (Taft's steric hindrance constant) greater than 1.75 as calculated from the values given for primary amines in Table V in D. F. DeTar, Journal of Organic Chemistry, 45, 5174 (1980), to which reference is made for a description of this parameter.

Another means for determining whether a secondary amino compound is “severely sterically hindered” is by measuring its ¹⁵N nuclear magnetic resonance (NMR) chemical shift. It has been found that the sterically hindered secondary amino compounds have a ¹⁵N NMR chemical shift greater than about δ+40 ppm, when a 90% by wt. amine solution in 10% by wt. D₂O at 35° C. is measured by a spectrometer using liquid (neat) ammonia at 25° C. as a zero reference value. Under these conditions, the tertiary amino compound used for comparison, methyldiethanolamine, has a measured ¹⁵N NMR chemical shift value of δ27.4. For example, 2-(2-tertiarybutylamino) propoxyethanol, 3-(tertiarybutylamino)-1-propanol, 2-(2-isopropylamino)-propoxyethanol and tertiarybutylaminoethoxyethanol had measured ¹⁵N NMR chemical shift values of δ+74.3, δ+65.9, δ+65.7 and δ+60. 5 ppm, respectively, whereas the ordinary sterically hindered amine, secondary-butylaminoethoxyethanol and the non-sterically hindered amine, n-butylaminoethoxyethanol had measured ¹⁵N NMR chemical shift values of .δ+48.9 and δ35.8 ppm, respectively. When the cumulative Es values is plotted against the ¹⁵N NMR chemical shift values of the amino compounds mentioned above, a straight line is observed. Amino compounds having an 15N NMR chemical shift values greater than δ+50 ppm under these test conditions had a higher H₂S selectively than those amino compounds having an ¹⁵N NMR chemical shift less than δ+50 ppm.

### Antioxidants

While the co-solvents may be used with the classes of antioxidant which are soluble to an adequate degree in the aqueous amino solution, the effect of the co-solvent is likely to be most useful with those that are not. While the chemistries of both the soluble and effectively insoluble (i.e., insoluble to any significant extent such that their functionality in the aqueous solution is impaired) may vary widely, they are likely to fall into several broad classes, namely, phenolic, aminic and esters including typically, amines especially aromatic amines, diamines, hydroxylamines and hydrazines, phenols, phosphites. Representative of such antioxidants are:
diphenylamine
dinaphthyl amine
phenyl-alpha naphthyl amine
butyl-alpha naphthyl amine
phenyl-beta naphthyl amine
ditolyl amine
phenyl tolyl amine
tolyl naphthyl amine
dioctyl diphenyl amine
dicyclohexyl amine
diphenyl p-phenylene diamine
mixtures of mono- and di-heptyl diphenylamines
4-tertiary butyl catechol
2,4-ditertiary butyl p-cresol
2,6-ditertiary butyl-4-methyl phenol hexyl gallate
tritertiary amyl phenyl phosphite
polymerized trimethyl dihydroquinoline
phenothiazine.

The following are exemplary classes of phenolic compounds:
1. Single 2,6-dialkylphenols, such as 2,6-di-tert.-butyl-4-methylphenol, 2,6-di-tert.-butyl-4-methoxymethylphenol or 2,6-di-tert.-butyl-4-methoxyphenol.
2. Bisphenols, such as 2,2′-methylene-bis-(6-tert.-butyl-4-methylphenol), 2,2′-methylene-bis-(6-tert.-butyl-4-ethylphenol), 2,2′-methylene-bis-4-methyl-6-α-methylcyclohexyl)-phenol!, 1,1-bis-(5-tert.-butyl-4-hydroxy-2-methylphenyl)-butane, 2,2-bis-(5-tert.-butyl-4-hydroxy-2-methylphenyl)-butane, 2,2-bis-(3,5-di-tert.butyl-4-hydroxyphenyl)-propane, 1,1,3-tris-(5-tert.butyl-4-hydroxy-2-methylphenyl)-butane, 2,2-bis-(5-tert.-butyl-4-hydroxy-2-methylphenyl)-4-n-dodecylmercapto-butane, 1,1,5,5-tetra-(5-tert.-butyl-4-hydroxy-2-methylphenyl)-pentane, ethylene glycol-bis-3,3-bis-(3′-tert.-butyl-4′-hydroxyphenyl)-butyrate!, 1,1-bis-(3,5-dimethyl-2-hydroxyphenyl)-3-(n-dodecylthio)-butane, or 4,4′-thio-bis-(6-tert.-butyl-3-methylphenol).
3. Hydroxybenzyl aromates, such as 1,3,5-tri-(3,5-di-tert.-butyl-4-hydroxybenzyl)-2,4,6-trimethylbenzene, 2,2-bis-(3,5-di-tert.-butyl-4-hydroxybenzyl)-malonic acid-dioctadecyl ester, 1,3,5-tris-(3,5-di-tert.-butyl-4-hydroxybenzyl)-isocyanurate, or 3,5-di-tert.-butyl-4-hydroxybenzyl-phosphonic acid-diethyl ester.
4. Amides of β-(3,5-di-tert.-butyl-4-hydroxyphenyl)-propionic acid, such as 1,3,5-tris-(3,5-di-tert.-butyl-4-hydroxyphenyl-propionyl)-hexahydro-s-triazine, i-(3,5-di-tert.-butyl-4-hydroxy-phenyl-propionyl)-hexamethylenediamine.
5. Esters of β-(3,5-di-tert.butyl-4-hydroxyphenyl)-propionic acid with mono- or polyvalent alcohols, such as with methanol, octadecanol, 1,6-hexanediol, ethylene glycol, thiodiethylene glycol, neopentyl glycol, pentaerythritol, tri-hydroxyethyl-isocyanurate.
6. Spiro compounds, such as diphenolic spiro-diacetals or spiro-diketals, such as 2,4,8,10-tetraoxaspiro-5,5!-undecane substituted in the 3- and 9-position with phenolic radicals, such as 3,9-bis-(3,5-di-tert.butyl-4-hydroxyphenyl)-2,4,8,10-tetraoxaspiro-5,5!-undecane, 3,9-bis-1,1-dimethyl-2-(3,5-ditert.-butyl-4-hydroxyphenyl)-ethyl!-2,4,8,10-tetraoxaspiro-5,5!-undecane.

Individual phenolic antioxidants may include:
1,3,5-tri-(3,5-di-tert.-butyl-4-hydroxybenzyl)-2,4,6-trimethylbenzene,
Pentaerythritol-tetra 3-(3,5-di-tert.-butyl-4-hydroxyphenyl)-propionate,
β-(3,5-di-tert.-butyl-4-hydroxyphenyl)-propionic acid-n-octadecyl ester,
thiodiethylene glycol-β-4-hydroxy-3,5-di-tert.-butyl-phenyl!-propionate, and
2,6-di-tert.-butyl-4-methyl-phenol

Aminic antioxidants may include, for example:
1. Aminoaryl derivatives, e.g., phenyl-1-naphthylamine, phenyl-2-naphthylamine, N,N′-diphenyl-p-phenylenediamine, N,N′-di-2-naphthyl-p-phenylenediamine, N,N′-di-2-naphthyl-p-phenylenediamine, N,N′-di-sec.-butyl-p-phenylenediamine, 6-ethoxy-2,2,4-trimethyl-1,2-dihydroquinoline, 6-dodecyl-2,2,4-trimethyl-1,2-dihydroquinoline, mono- and dioctyliminodibenzyl, polymerised 2,2,4-trimethyl-1,2-dihydroquinoline. Octylated diphenylamine, nonylated diphenylamine, N-phenyl-N′-cyclohexyl-p-phenylenediamine, N-phenyl-N′-isopropyl-p-phenylenediamine, N,N′-di-sec.octyl-p-phenylenediamine, N-phenyl-N′-sec.-octyl-p-phenylenediamine, N,N′-di-(1,4-dimethylpentyl)-p-phenylenediamine, N,N′-dimethyl-N,N′-di-(sec.-octyl)-p-phenylenediamine, 2,6-dimethyl-4-methoxyaniline, 4-ethoxy-N-sec.-butylaniline, diphenylamineacetone condensation product, aldol-1-naphthylamine and phenothiazine.
2. Sterically hindered amines, e.g., 4-benzoyl-2,2,6,6-tetramethylpiperidine, 4-stearoyloxy-2,2,6,6-tetramethylpiperidine, bis-(2,2,6,6-tetramethylpiperidyl)-sebacate or 3-n-octyl-7,7,9,9-tetramethyl-1,3,8-triaza-spiro 4,5 !decane-2,4-dione.

### Antioxidant/Amine Co-Solvent

The co-solvent should be selected by empirical means in dependence on the identity of the specific antioxidant and amine used in the aqueous gas treating solution. Generally, however, the co-solvent will exhibit mixed hydrophilic and lipophilic character. A hydrophilic/lipophilic balance (HLB) from 7 to 12, preferably from 8 to 11 with optimal results likely to accrue at about 10. If the risk of some degree of foaming can be accepted an HLB of about 12 to 15 may be found suitable.

One important consideration in the selection of the co-solvent is that it should not be excessively volatile during the regeneration step in temperature swing operation or in pressure swing operation where reduced pressure and agitation in the regeneration column may favor evaporation. Normally, the temperature will be above 100° C. although lower temperatures may be used with certain absorbent systems. This means that the co-solvent should have a boiling point above 100° C. and preferably above 120° C. or higher. With this factor in play co-solvent such as the following may be useful depending on the amine system and the additive used in it: alcohols such as 1-butanol, b.p. 118° C., 1-hexanol, b.p. 155-159° C., 1-octanol, b.p. 195° C., ethylene glycol, b.p. 197.3° C., propylene glycol b.p. 188.2° C. diethylene glycol b.p. 244-24° C.; triethylene glycol b.p. 285° C., dipropylene glycol b.p. 230.5° C. and higher polyethylene and polypropylene glycols. Ethers including the ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether and diethylene glycol mono-n-butyl ether have properties commending consideration of their use as co-solvents. Esters, having both hydrophilic and oleophilic groups are also likely to be useful, for example, ethyl butyrate, ethyl hexanaote, triethylene glycol hexanoate.

1. A process for the selective absorption of normally gaseous acidic components from normally gaseous hydrocarbon mixtures containing both the acidic component and gaseous non-acidic components which comprises circulating an aqueous amine absorbent solution comprising an amine absorbent, at least one antioxidant and a co-solvent for the amine and the antioxidant in a cyclic amine absorption gas purification unit to absorb acidic gases from the hydrocarbon gas mixture in an absorption tower and desorbing acidic gases from the absorbent solution in a regeneration tower to produce a stream of purified hydrocarbon gas and at least one stream of acidic gas removed from the hydrocarbon gas.

2. A process according to claim 1 in which the gaseous mixture is contacted in countercurrent with the absorbent solution in the absorption tower at an inlet temperature from 20° to 100° C. and the absorbent solution containing absorbed acidic component(s) is regenerated in the regeneration tower at a temperature from 50° to 170° C.

3. A process according to claim 2 in which the gaseous mixture is contacted in countercurrent with the absorbent solution in the absorption tower at an inlet temperature from 40° to about 60° C. and is regenerated at a temperature from 80° to 120° C.

4. A process according to claim 1 in which the amine absorbent has the formula: 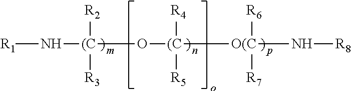
where R¹ and R⁸ are each C1 to C8 alkyl and C2 to C8 hydroxyalkyl groups, R², R³, R⁴, R⁵, R⁶, and R⁷ are each hydrogen, C₁-C₄ alkyl and hydroxyalkyl groups, with certain provisos to define the adequately hindered molecule and m, n, and p are integers from 2 to 4 and o is zero or an integer from 1 to 10.

5. A process according to claim 1 in which the amine absorbent has the formula: 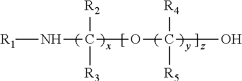
where R¹ is C₁-C₈ primary alkyl and primary C₂-C₈ hydroxyalkyl, C₃-C₈ branched chain alkyl and branched chain hydroxyalkyl and C₃-C₈ cycloalkyl and hydroxycycloalkyl, R², R³, R⁴ and R⁵ are each hydrogen, C₁-C₄ alkyl and C₁-C₄ hydroxyalkyl radicals, with the proviso that when R¹ is a primary alkyl or hydroxyalkyl radical, both R² and R³ bonded to the carbon atom directly bonded to the nitrogen atom are alkyl or hydroxyalkyl radicals and that when the carbon atom of R¹ directly bonded to the nitrogen atom is secondary at least one of R² or R³ bonded to the carbon atom directly bonded to the nitrogen atom is an alkyl or hydroxyalkyl radical, x and y are each positive integers from 2 to 4 and z is an integer from 1 to 4.

6. A process according to claim 1 in which the amine comprises a diamino ether and an aminoether alcohol represented by the respective formulae: 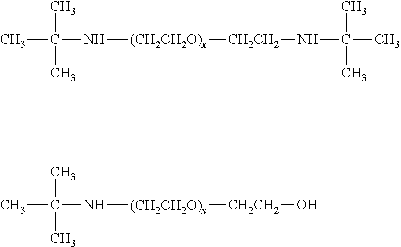
where x is an integer ranging from 2 to 6.

7. A process according to claim 1 in which the amine comprises a diamino ether and aminoalcohol represented by the respective formulae: 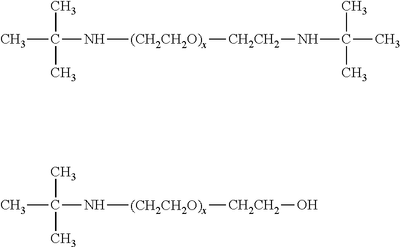
where x is an integer ranging from 2 to 6 and the weight ratio of the first amine to the second amine ranging from 0.23:1 to 2.3:1 and preferably 0.43 to 2.3:1.

8. A process according to claim 1 in which the amine comprises a mixture of bis-(tert-butylaminoethoxy) ethane and tert-butylaminoethoxyethoxyethanol.

9. A process according to claim 1 in which the amine comprises a compound of the formula:
R¹—NH—[CnH2n—O-]x—OY
where R¹ is a secondary or tertiary alkyl group of 3 to 8 carbon atoms, preferably a tertiary group of 4 to 8 carbon atoms, Y is H or alkyl of 1 to 6 carbon atoms, n is a positive integer from 3 to 8 and x is a positive integer from 3 to 6.

10. A process according to claim 1 in which the antioxidant comprises an aromatic amine, an aromatic diamine, a phenol or a phosphite ester.

11. A process according to claim 1 in which the co-solvent has mixed hydrophilic and lipophilic character with a hydrophilic/lipophilic balance (HLB) from 7 to 15.

12. A process according to claim 11 in which the co-solvent has mixed hydrophilic and lipophilic character with a hydrophilic/lipophilic balance (HLB) from 8 to 11.

13. A process according to claim 1 in which the co-solvent has a boiling point above 100° C.

14. A process according to claim 13 in which the co-solvent has a boiling point above 120° C.

15. A process according to claim 1 in which the co-solvent comprises 1-butanol, 1-hexanol, 1-octanol, ethylene glycol, propylene glycol, diethylene glycol; triethylene glycol, dipropylene glycol, ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether, diethylene glycol mono-n-butyl ether, ethyl butyrate, ethyl hexanaote or triethylene glycol hexanoate.

16. An absorbent formulation for the selective absorption of normally gaseous acidic components from normally gaseous hydrocarbon mixtures containing both the acidic component and gaseous non-acidic components, comprising (i) an amine absorbent for the selective absorption of normally gaseous acidic components from hydrocarbon gas mixtures containing the acidic component and gaseous non-acidic components, (ii) an antioxidant and (iii) a co-solvent for the amine and the antioxidant.

17. An absorbent formulation according to claim 16 in which the antioxidant comprises an aromatic amine, an aromatic diamine, a phenol or a phosphite ester.

18. An absorbent formulation according to claim 16 in which the co-solvent has mixed hydrophilic and lipophilic character with a hydrophilic/lipophilic balance (HLB) from 7 to 15.

19. An absorbent formulation according to claim 16 in which the co-solvent has mixed hydrophilic and lipophilic character with a hydrophilic/lipophilic balance (HLB) from 8 to 11.

20. An absorbent formulation according to claim 16 in which the co-solvent has a boiling point above 100° C.

21. An absorbent formulation according to claim 20 in which the co-solvent has a boiling point above 120° C.

22. An absorbent formulation according to claim 16 in which the co-solvent comprises 1-butanol, 1-hexanol, 1-octanol, ethylene glycol, propylene glycol, diethylene glycol; triethylene glycol, dipropylene glycol, ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether, diethylene glycol mono-n-butyl ether, ethyl butyrate, ethyl hexanaote or triethylene glycol hexanoate.
